(19) United States
(12) Patent Application Publication
(10) Pub. No.: US 2024/0115658 A1
(43) Pub. Date: Apr. 11, 2024

(54) Use of an intervertebral disc fibrosis-inducing substance in preparation of medication

(71) Applicant: Shanghai Ninth People's Hospital, Shanghai JiaoTong University School of Medicine, Shanghai (CN)

(72) Inventors: Jie ZHAO, Shanghai (CN); Chen CHEN, Shanghai (CN); Xiao YANG, Shanghai (CN); Tangjun ZHOU, Shanghai (CN); An QIN, Shanghai (CN); Zhiqian CHEN, Shanghai (CN); Kai ZHANG, Shanghai (CN); Yifan ZHOU, Shanghai (CN)

(73) Assignee: Shanghai Ninth People's Hospital, Shanghai JiaoTong University School of Medicine, Shanghai (CN)

(21) Appl. No.: 18/029,112

(22) PCT Filed: Jun. 11, 2021
(86) PCT No.: PCT/CN2021/099562

§ 371 Date: Nov. 6, 2023

(30) Foreign Application Priority Data
Sep. 29, 2020 (CN) 2020110473219

(51) Int. Cl. A61K 38/14; A61K 35/33; A61P 19/02

(52) U.S. Cl. CPC A61K 38/14; A61K 35/33; A61P 19/02

(57) ABSTRACT

A use of an intervertebral disc fibrosis-inducing substance in preparation of medication, where the medication is used for the treatment of degenerative spinal diseases. The inventors of the present disclosure make use of the pro-fibrotic effect of the intervertebral disc fibrosis-inducing substance and the auxiliary effect of the fibroblast to achieve further treatment of intervertebral disc degeneration. The simultaneous administration of the above two drugs can reasonably control the degeneration and achieve the purpose of stepped treatment, thereby having clinical transformation value. In addition, the therapy combining the intervertebral disc fibrosis-inducing substance with the cell in the present disclosure allows the treatment regimen more personalized.

### FIELD OF THE INVENTION

The present disclosure relates to the field of medicine, particularly to a use of an intervertebral disc fibrosis-inducing substance in preparation of medication.

### BACKGROUND OF THE INVENTION

About 600 million people in today's society suffer from low back pain due to intervertebral disc degeneration, and the treatment of this degenerative disease causes a huge burden on the medical and socioeconomic systems of all developing and developed countries. Intervertebral disc degeneration is a complex process and the intervertebral disc consists of three major components which are nucleus pulposus (NP), annulus fibrous (AF), and two cartilage endplates, respectively. The nucleus pulposus is a colloid center of the disc and rich in proteoglycan, the annulus fibrous is a partially concentric fibrocartilage tissue rich in collagen and surrounds the nucleus pulposus, and the two cartilage endplates are in junction with the upper and lower vertebral bodies, respectively. Intervertebral disc degeneration is a progressive cell-mediated molecular cascade reaction involving structural and biomechanical changes, where the proteoglycan content of the nucleus pulposus decreases and the ratio of the proteoglycan to collagen decreases, resulting in a loss of hydrostatic performance of the nucleus pulposus. This structural wear, which is also a biomechanical impairment, leads to continuous dehydration and collapse of the nucleus pulposus, loss of disc height, and gradual loss of the boundary between the nucleus pulposus and the annulus fibrosus, ultimately leading to complete intervertebral disc degeneration. In severe cases, patients may experience spinal instability and nerve root compression which result in chronic back pain.

The intervertebral disc is weak in self-repair and regeneration, which may be due to the low number of disc cells and lack of nutrient supply to the tissue. Therefore, conventional treatment strategies can merely realize symptom relief and have limited long-term efficacy. Surgical intervention is usually performed to restore the disc height, maintain the stability of the intervertebral space, and relieve severe neurological symptoms. However, spinal fusion can not restore biomechanical properties, and increased mechanical stress between adjacent vertebrae may instead cause them to degenerate.

### SUMMARY OF THE INVENTION

The present disclosure provides a use of an intervertebral disc fibrosis-inducing substance in preparation of medication, where the medication is used for the treatment of degenerative spinal diseases.

In some embodiments of the present disclosure, the intervertebral disc fibrosis-inducing substance comprises bleomycin.

In some embodiments of the present disclosure, the intervertebral disc fibrosis-inducing substance comprises a combination of bleomycin and fibroblast.

In some embodiments of the present disclosure, the fibroblast is a dermal fibroblast. In some embodiments of the present disclosure, the fibroblast is an autologous fibroblast.

In some embodiments of the present disclosure, the intervertebral disc fibrosis-inducing substance is capable of inducing the high TGF-β expression of annulus fibrosus cells.

In some embodiments of the present disclosure, the intervertebral disc fibrosis-inducing substance is capable of upregulating the expression of p-Smad2 and/or p-Samd3 in annulus fibrosus cells.

In some embodiments of the present disclosure, the intervertebral disc fibrosis-inducing substance is capable of upregulating the expression of FSP1 in annulus fibrosus cells.

In some embodiments of the present disclosure, the intervertebral disc fibrosis-inducing substance is capable of upregulating the expression of type I collagen in annulus fibrosus cells.

In some embodiments of the present disclosure, the intervertebral disc fibrosis-inducing substance is capable of upregulating the expression of type III collagen in annulus fibrosus cells.

In some embodiments of the present disclosure, the degenerative spinal disease is selected from intervertebral disc degeneration.

In some embodiments of the present disclosure, the intervertebral disc fibrosis-inducing substance is the only active ingredient.

### BRIEF DESCRIPTION OF DRAWINGS

FIG. 1 is a schematic diagram showing experimental results in Embodiment 1, where FIG. 1A and FIG. 1B show results of bleomycin stimulation of fibroblasts and activation of TGFβ/Smad pathway, and FIG. 1C shows an immunoprotein western-blot of highly expressed fibrogenic-associated proteins TGFβ, FSP1, and Col1a1 induced by bleomycin.

FIG. 2 is a schematic diagram showing results of disc local injection of bleomycin for spinal degeneration treatment in Embodiment 3, where FIG. 2A is a schematic diagram showing results of X-ray, and FIG. 2B is a schematic diagram showing results of MRI.

FIG. 3 is a schematic diagram showing results of safranin O-fast green staining and Sirius red staining of paraffin sections after six weeks of disc local injection of bleomycin for spinal degeneration treatment in Embodiment 4.

FIG. 4 is a schematic diagram showing quantitative statistical results of safranin O-fast green staining and Sirius red staining after six weeks of disc local injection of bleomycin for spinal degeneration treatment in Embodiment 4.

FIG. 5 is a schematic diagram showing quantitative statistical results of TGFβ1, TGFβR1, Col1a1, and FSP1 immunofluorescence staining after six weeks of disc local injection of bleomycin for spinal degeneration treatment in Embodiment 4.

FIG. 6 is a schematic diagram showing quantitative statistical results of TGFβ1, TGFβR1, Col1a1, and FSP1 immunofluorescence staining after six weeks of disc local injection of bleomycin for spinal degeneration treatment in Embodiment 4.

FIG. 7 is a schematic diagram showing atomic force analysis results after six weeks of disc local injection of bleomycin for spinal degeneration treatment in Embodiment 5.

FIG. 8 is a schematic diagram showing quantitative statistics results of the atomic force analysis in Embodiment 5.

FIG. 9 is a schematic diagram showing results of bleomycin+fibroblast disc local injection for spinal degeneration treatment in Embodiment 7, where FIG. 9A is a schematic diagram showing results of X-ray, and FIG. 9B is a schematic diagram showing results of MRI.

### DETAILED DESCRIPTION OF THE INVENTION

To clarify the purposes, technical solutions, and beneficial technical effects of the present disclosure, further description is provided below with reference to specific embodiments. For those of ordinary skill in the art, other advantages and efficacy may be obtained based on the specification as disclosed herein.

A first aspect of the present disclosure provides a use of an intervertebral disc fibrosis-inducing substance in preparation of medication, where the medication is used for the treatment of degenerative spinal diseases. The inventors of the present disclosure have found that intervertebral disc fibrosis-inducing substances (e.g., bleomycin, etc.) can be directly used in the treatment of degenerative spinal disorders on the principle of maintaining or even restoring intervertebral space height, preventing nerve root compression and spinal instability, and achieving rapid and sufficient fibrosis, so that timely stabilization can be realized and degenerative spinal disorders can be effectively controlled.

In the present disclosure, the intervertebral disc fibrosis-inducing substances can usually be administered to (between) the intervertebral discs, so as to induce the migration of disc annulus fibrosus cells, secretion and deposition of fibrotic collagen, thereby providing effective support to maintain the intervertebral space height, preserving a certain degree of vertebral mobility, maintaining intervertebral stability, and preventing further degenerative changes. Suitable methods of administering the above-mentioned intervertebral disc fibrosis-inducing substances to individuals are known to those skilled in the art, for example, the intervertebral disc fibrosis-inducing substances can be administered in a form of injection and the like. In a specific embodiment of the present disclosure, bleomycin (a water-soluble crystalline substance) may be in a form of an aqueous solution and administered locally to the intervertebral discs as an injection. In another specific embodiment of the present disclosure, bleomycin may be in the form of an aqueous solution in which fibroblasts are dispersed and the aqueous solution may be administered locally to (between) the intervertebral discs as an injection.

In some embodiments of the present disclosure, the intervertebral disc fibrosis-inducing substance comprises bleomycin. Bleomycin is a well-known anticancer agent for clinical use and is a cytotoxic chemotherapeutic compound commonly used for the treatment of lymphomas, leukemias, squamous cell carcinomas, and some genital tract tumors. In a specific embodiment of the present disclosure, bleomycin can effectively prompt annulus fibrosus cells to sustainedly release or highly express TGFβ, upregulate the expression of p-Smad2 and/or p-Samd3 in annulus fibrosus cells, and upregulate the expression of type I collagen and/or type III collagen in annulus fibrosus cells, thereby effectively inducing intervertebral disc fibrosis and to a certain extent restoring intervertebral disc height and biomechanical properties.

In the present disclosure, the intervertebral disc fibrosis-inducing substance further comprises a fibroblast. Specifically, the intervertebral disc fibrosis-inducing substance comprises a combination of bleomycin and the fibroblast. Fibroblasts, also known as fibroblast cells, are usually the main cellular component of loose connective tissues and are terminally differentiated cells that secrete a large number of type I collagen fibers.

In the present disclosure, the fibroblast may be a dermal fibroblast which is mainly derived from the dermis of the skin. Specifically, the skin covers the surface of the body and is composed of the epidermis, dermis, and subcutaneous layer in order from the outside to the inside, where the dermis is mainly composed of fibroblasts and the fibers and stroma produced by them. The fibroblast may be allogeneic and/or xenogeneic, or homogeneous, preferably, the fibroblast may be autogenous, so that rejection of the body can be effectively avoided to ensure better safety. In a specific embodiment of the present disclosure, the combination of bleomycin and the fibroblast can more effectively prompt annulus fibrosus cells to sustainedly release or highly express TGFβ, upregulate the expression of p-Smad2 and/or p-Samd3 in annulus fibrosus cells, and upregulate the expression of type I collagen and/or type III collagen in annulus fibrosus cells, thereby more effectively inducing intervertebral disc fibrosis and restoring intervertebral disc height and biomechanical properties.

In the present disclosure, the intervertebral disc fibrosis-inducing substance may serve as a promoter of transforming growth factor-β (TGF-β), for example, a promoter of transforming growth factor-β in annulus fibrosus cells. The intervertebral disc fibrosis-inducing substance is capable of inducing a sustained release or high expression of TGF-β in annulus fibrosus cells, thereby avoiding repeated administration. In a specific embodiment of the present disclosure, bleomycin or the combination of bleomycin and the fibroblast can activate the TGF-β/Smad pathway in annulus fibrosus cells and induce Smad2/3 phosphorylation, thereby allowing high expression of TGFβ.

In the present disclosure, the intervertebral disc fibrosis-inducing substance may serve as a promoter of fibroblast specific protein 1 (FSP1), for example, a promoter of fibroblast specific protein 1 in annulus fibrosus cells. The intervertebral disc fibrosis-inducing substance may induce an increase in FSP1 expression in annulus fibrosus cells. In a specific embodiment of the present disclosure, bleomycin or the combination of bleomycin and the fibroblast can activate the TGF-β/Smad pathway in annulus fibrosus cells and induce Smad2/3 phosphorylation, thereby increasing the expression of the end-protein product FSP1.

In the present disclosure, the intervertebral disc fibrosis-inducing substance may serve as a promoter of type I collagen, for example, a promoter of type I collagen in annulus fibrosus cells. The intervertebral disc fibrosis-inducing substance can upregulate the expression of type I collagen in annulus fibrosus cells. In a specific embodiment of the present disclosure, bleomycin or the combination of bleomycin and the fibroblast can stimulate annulus fibrosus cells and enable an increase in the expression of type I collagen.

In the present disclosure, the intervertebral disc fibrosis-inducing substance may serve as a promoter of type III collagen, for example, a promoter of type III collagen in annulus fibrosus cells. The intervertebral disc fibrosis-inducing substance can upregulate the expression of type III collagen in annulus fibrosus cells. In a specific embodiment of the present disclosure, bleomycin or the combination of bleomycin and the fibroblast can stimulate annulus fibrosus cells and enable an increase in the expression of type III collagen.

In the present disclosure, the intervertebral disc fibrosis-inducing substance may serve as a promoter of Smad2/3 phosphorylation, for example, a promoter of Smad2/3 phosphorylation in annulus fibrosus cells. The intervertebral disc fibrosis-inducing substance can upregulate the expression of p-Smad2 and/or p-Samd3 in annulus fibrosus cells. In a specific embodiment of the present disclosure, bleomycin or the combination of bleomycin and the fibroblast can activate the TGF-β/Smad pathway in annulus fibrosus cells and induce Smad2/3 phosphorylation, thereby allowing the simultaneous high expression of TGF-β and its downstream factors p-Smad2 and/or p-Samd3. It can be seen that the Smad2 and/or Samd3 phosphorylation in annulus fibrosus cells can be effectively induced and the expression of p-Smad2 and/or p-Samd3 in annulus fibrosus cells can be upregulated by high expression of TGF-β, thereby inducing intervertebral disc fibrosis.

In some embodiments of the present disclosure, the degenerative spinal disease may be intervertebral disc degeneration. Specifically, the degenerative spinal disease may be acute or subacute intervertebral disc degeneration, intervertebral space collapse, intervertebral disc protrusion, spinal stenosis, spondylolisthesis, and other diseases. Specifically, patients with degenerative spinal diseases may be those who clinically present with reduced disc height, but no curvature and collapse of the intervertebral space, no significant spinal stenosis, and no neurological symptoms after surgical or non-surgical treatment.

In some embodiments of the present disclosure, the intervertebral disc fibrosis-inducing substance is the only active ingredient.

A second aspect of the present disclosure provides a composition, comprising the intervertebral disc fibrosis-inducing substance, where the intervertebral disc fibrosis-inducing substance comprises a combination of bleomycin and fibroblast.

The composition provided by the present disclosure may further comprise at least one pharmaceutically acceptable carrier, which generally refers to carriers for administration. The pharmaceutically acceptable carrier does not induce the production of antibodies harmful to the individual receiving the composition and does not have excessive toxicity after administration. These carriers are well known to those skilled in the art, for example, related contents of pharmaceutically acceptable carriers as disclosed in Remington's Pharmaceutical Sciences (Mack Pub. Co., N. J. 1991). Specifically, the carrier may be one or more of saline, buffer, glucose, water, glycerol, ethanol, adjuvants, etc., and the carrier is not limited thereto. In the present disclosure, the “subject” typically includes humans and non-human primates (such as mammals, dogs, cats, horses, sheep, pigs, cattle, etc).

A third aspect of the present disclosure provides a treatment method, comprising: administering an effective amount of the above-described intervertebral disc fibrosis-inducing substance, or the composition provided in the second aspect of the present disclosure to a subject in need thereof. The “effective amount” usually refers to an amount that, after an appropriate administration period, achieves the desired effect, e.g., imaging, treatment of a disease, etc.

The inventors of the present disclosure make use of the pro-fibrotic effect of the intervertebral disc fibrosis-inducing substance and the auxiliary effect of the fibroblast to achieve further treatment of intervertebral disc degeneration. The simultaneous administration of the above two drugs can reasonably control the degeneration and achieve the purpose of stepped treatment, thereby having clinical transformation value. In addition, the therapy combining the intervertebral disc fibrosis-inducing substance with the cell in the present disclosure allows the treatment regimen more personalized.

The invention of the present disclosure is further described below through embodiments, and the scope of the present disclosure is not limited thereby.

Unless otherwise stated, the laboratory procedures, test methods, and production methods disclosed in the present disclosure are those well-known and commonly used in the field of molecular biology, biochemistry, chromatin biology, analytical chemistry, cell culture, recombinant DNA, and other routine techniques. These methods have been well described in the existing literature, and please refer to Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; and METHODS IN MOLECULAR BIOLOGY, Vol. 119, Chromatin Protocols (P. B. Becker, ed.) Humana Press, Totowa, 1999, etc.

### Embodiment 1

Activation of the TGF-β/Smad pathway in annulus fibrosus cells by bleomycin:

Method for immunoprotein western-blotting of annulus fibrosus cells:

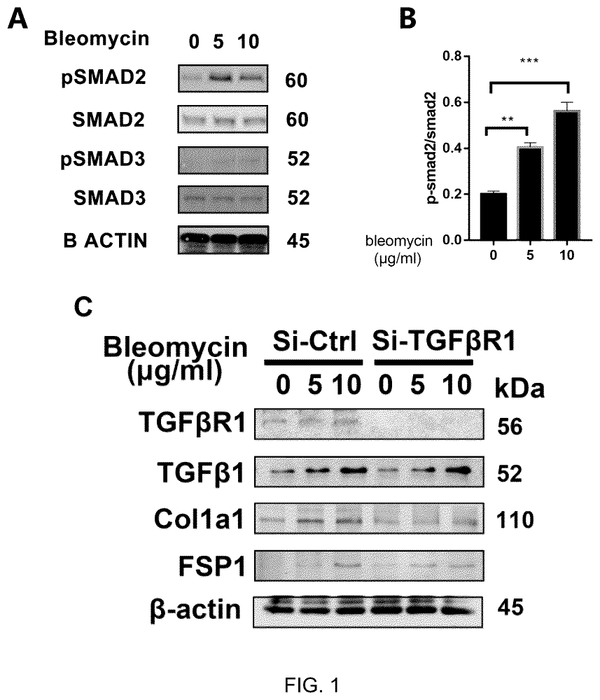

Annulus fibrosus cells (provided by CAS Cell Bank) were cultured in a six-well plate to which high-sugar DMEM medium+10% fetal bovine serum was added, the plate was placed in a 5% CO₂ incubator at 37° C., and 5, 10 μg/ml of bleomycin and/or the TGFβR1 inhibitor LY364947 was added when the cell confluence was 70%-80% to co-culture for 30 min or 24 h. Total cellular protein was extracted from cultured annulus fibrosus cells (Medical Center of Rush University) by using RIPA lysis buffer (Roche, Basel, Switzerland) containing phosphatase and protease inhibitors. Equal amounts (20-30 μg) of extracted proteins were obtained by using 10% or 12.5% SDSPAGE gel, and proteins were separated by electroblotting with 0.22 μm PVDF membrane (Merck Millipore). The PVDF membrane was blocked with 5% BSA-PBS for 1 h at room temperature and then incubated with primary antibody (diluted at a ratio of 1:1000 in 5% BSA-PBS) overnight (at least 16 h) at 4° C. Anti-SMAD2 antibody (Ser308, D4364; rabbit monoclonal antibody), anti-phosphorylated SMAD2 antibody (Ser465/467, 138D4; rabbit monoclonal antibody), anti-SMAD3 antibody (C67H9; rabbit monoclonal antibody), anti-phosphorylated SMAD3 antibody (Ser423/425, C25A9; rabbit monoclonal antibody), anti-SMAD2/3 antibody (D7G7; rabbit monoclonal antibody), anti-phospho SMAD2/3 antibody (Ser465/467; Ser423/425; rabbit monoclonal antibody), anti-SMAD4 antibody (D3M6U; rabbit monoclonal antibody), and anti-1-actin antibody (D6A8; rabbit monoclonal antibody) were purchased from Cell Signaling Technology (Danvers, Massachusetts, USA). Primary antibodies of FSP1 antibody (S100A4; rabbit monoclonal antibody), transforming growth factor β receptor I (ab31013; rabbit monoclonal antibody), type I collagen (ab6308; rabbit monoclonal antibody), and transforming growth factor β1 (ab64715; rabbit monoclonal antibody) were purchased from Abcam (Cambridge, UK). Membranes were then rinsed in Tris buffered saline-Tween 20 (TBST), followed by anti-rabbit IgG (H+L) (DyLight) Culture™ 8004×PEG conjugate; Cell Signaling Technology) secondary antibody (1:5000 dilution) for 1 hour at room temperature in the dark. The membrane was extensively rinsed in TBST again and protein immunobands were detected by using the LI-COR Odyssey Fluorescence Imaging System (LICOR Biosciences, Lincoln, NE, USA). It can be seen that p-smad2 and p-smad3 in annulus fibrosus cells were activated (as shown in FIG. 1A and FIG. 1B) in response to bleomycin stimulation, and the expression of end-protein products Col1a1 and FSP1 increased (as shown in FIG. 1C).

### Embodiment 2

All animal experiments were approved by the Animal Care and Ethics Committee of the Ninth People's Hospital of Shanghai Jiao Tong University of Medicine, and were conducted according to the principles and procedures of Guide for the Care and Use of Laboratory Animals of the National Institutes of Health (NIH). Six 8-week-old male Sprague-Dawley rats (Shanghai Laboratory, Shanghai Animal Research Center, China) were raised at 26-28° C. and 50-65% humidity for 12 h day and night. Standard rodent foods are fed to rats and the rats have free access to fresh water. Before surgery, the rats were anesthetized by intraperitoneal injection of sodium pentobarbital (5 mg/100 g of body weight). The tails of rats were disinfected with iodinated polyvinyl pyrrolidone, and then a longitudinal skin incision was made to the tail near the ventral part to expose 6-10 parts of the intervertebral disc of the tail bone. The Co6/7 disc was used as a sham control, and Co7/8, Co8/9, and Co9/10 were used as experimental groups. A sterile 20-gauge needle was used to perpendicularly puncture the intervertebral disc through the skin, so as to ensure the needle passed through the annulus fibrosus and stuck into the nucleus pulposus located at the center of the intervertebral disc. The incision was then sutured and the rats were allowed to recover for two weeks. The rats (n=6) were re-subjected to surgery to expose Co8/9 and Co9/10 discs, and 5 μl of 10 and 5 μg/ml bleomycin were injected into each disc. The surgical incision was then sutured and the rats were allowed to recover for four weeks. At the end of the experiment, all rats were executed, the tails were collected, the soft tissues were cleared, and the spines were fixed with 4% PFA. Co6/7 was the control group (sham group), Co7/8 was the Puncture group, and Co8/9 and Co9/10 were the puncture+bleomycin administration groups (Bleomycin group).

### Embodiment 3

Digital X-ray imaging of the punctured disc samples (Puncture and Bleomycin groups) prepared in Embodiment 2 was performed in the anterior-posterior axis with a detector that provides up to 5× geometric magnification (Faxitron VersaVision; Faxitron Bioptics LLC, Tucson, AZ. USA). MRI imaging of the same punctured disc samples was performed on a Siemens MagnetomPrisma E11 (Siemens Healthineers, Erlangen, Germany) with the following parameters: TR 3000 ms, TE 80 ms, thickness 1.1 mm, interval 0.22 mm, field of view 160×65 mm, voxel size 0.25×0.1 mm.

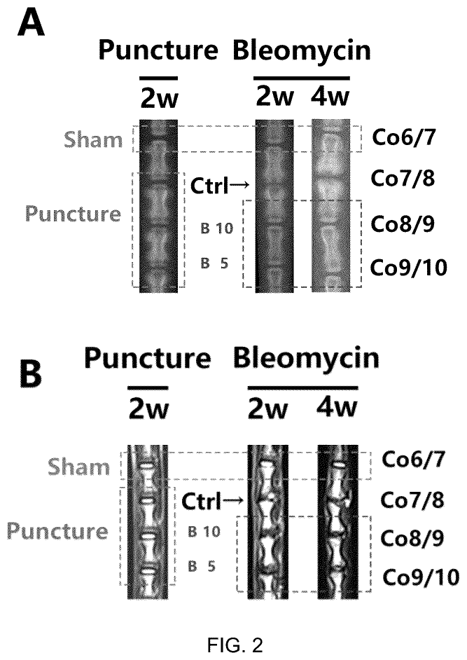

As seen from the digital X-ray imaging results, the intervertebral disc in the Puncture group was severely collapsed with osteophyma formation (shown on the left of FIG. 2A), and the signal of the intervertebral disc in the puncture group was darkened as seen from the MRI imaging results (shown on the left in FIG. 2B). The digital X-ray and MRI results of the corresponding bleomycin administration group (Bleomycin group) showed that the intervertebral space height could be effectively maintained after bleomycin treatment, and the stability of the disc was not significantly affected, although the signal of the intervertebral disc was difficult to be preserved (shown on the right of FIG. 2A and FIG. 2B).

### Embodiment 4

Fixed disc tissue specimens (sham group, Puncture group, and Bleomycin group) were embedded in paraffin blocks, followed by tissue sectioning (with a thickness of 5 μm). For histological evaluation, paraffin sections were stained with saffron O-solid green and Sirius red according to standard laboratory protocols. For immunofluorescence detection, tissue sections were de-paraffined in graded xylene, rehydrated in graded alcohol solution, and then incubated in antigen retrieval buffer (Roche) at 37° C. for 30 min. After cooling to room temperature, the slides were immersed in PBS (pH 7.4) and washed 3 times for 5 min each. Autofluorescence quencher was added to the sections, and 5 min later, blocking buffer was added to block the sections for 30 min at room temperature. The sections were then incubated with primary antibodies overnight in a wet chamber at 4° C. Primary antibodies were diluted at a ratio of 1:100, and included anti-Col1a1, anti-FSP1, anti-TGFβ, and anti-TGFβR1 (all purchased from Cell Signaling Technology). The next day, sections were washed with PBS, conjugated to secondary antibody (anti-rabbit, 1:500; Cell Signaling Technology) through Alexa-fluor594, and then incubated for 50 min in the dark at room temperature. The sections were washed with PBS and then incubated with DAPI solution (Sigma Aldrich, St. Louis, MO, USA) for 10 minutes in the dark to stain the nucleus. The sections were subjected to a final PBS wash, air-dried, and then sealed with an anti-fluorescence quenching flake. Digital fluorescence images were taken by using a Leica DM4000 B epifluorescence microscope (Leica Microsystems).

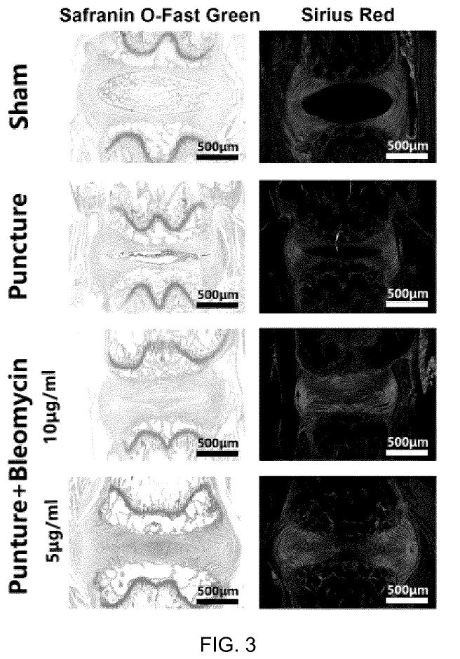

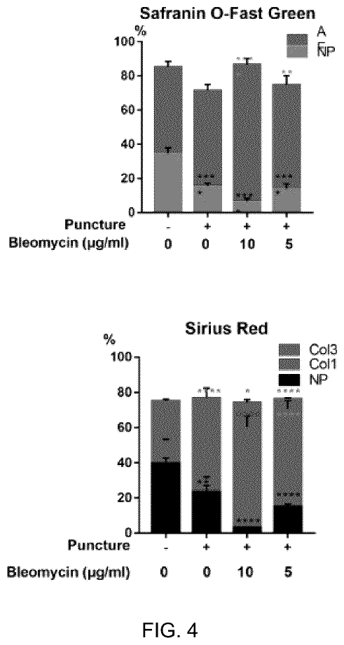

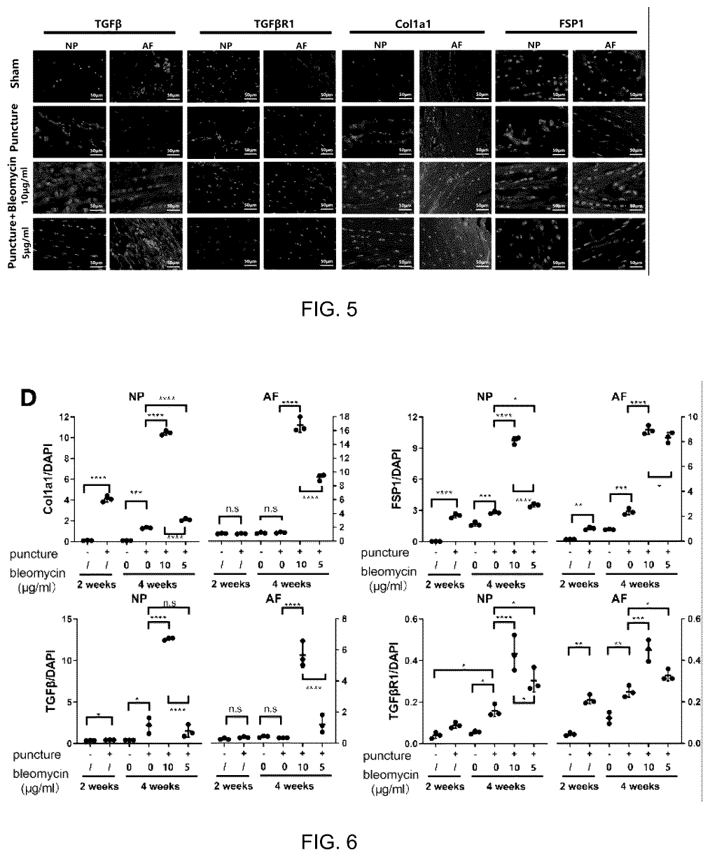

Safranin O-fast green staining and Sirius red staining results are shown in FIG. 3. In the Puncture group, the nucleus pulposus was dislodged, the annulus fibrous was disrupted, the intervertebral space collapsed, and the cartilage endplates severely degenerated. However, in the bleomycin administration group (Puncture+Bleomycin), the intervertebral space height was significantly higher, the annulus fibrosus was completely repaired and migrated inward, and large amounts of type I collagen were secreted to fill the intervertebral space. Statistical analysis showed that the staining percentages of annulus fibrous (green), type I collagen (yellow), and type III collagen (green) in the bleomycin administration group (Puncture+, Bleomycin 10 or 5) injected with 10 and 5 μg/ml of bleomycin were significantly increased compared with the puncture group (Puncture+, Bleomycin 0) and the control group (Puncture-, Bleomycin 0), which was shown as FIG. 4. Immunofluorescence results showed that a large amount of type I and type III collagens was secreted in the bleomycin administration group (Puncture+Bleomycin) (Puncture+, Bleomycin 10 or 5) (shown in FIGS. 5 and 6), indicating that bleomycin enables rapid and stable intervertebral disc fibrosis and secretion of a large amount of type I and type III collagen to fill the intervertebral space.

### Embodiment 5

Atomic force analysis was performed by first extracting and dissecting the punctured vertebrae. Paraffin sections were made and then nanoindented (R<10 nm, nominal N/m, tips: Si/Tipless/Top, cantilevered Si/Al/Top; Park Systems) by using a Park NX20 (Park Systems, Korea) equipped with a microsphere colloidal tip. For undulating surfaces with large areas, a scan rate of 13 hz was used. Large scanning rates can reduce drift, but are generally only used to scan small planes. Indentation was performed by using a scanning Asyst air probe, where a z-piezo displacement rate was 10 μm/s, a maximum load was ˜120 nn, a curvature radius was 5 nm, and a force constant was 0.4 N/m.

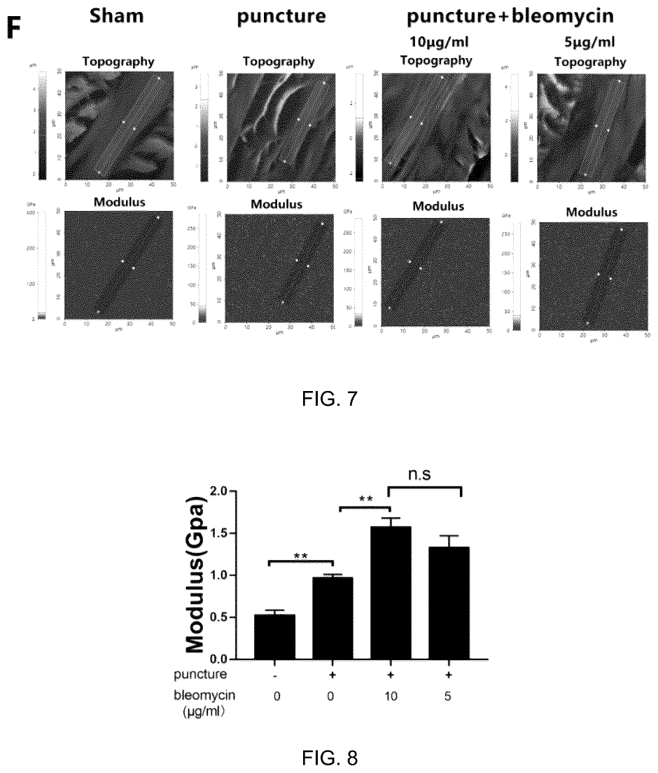

As can be seen from the atomic force analysis results in FIG. 7, there was an increase in the strain of annulus fibrosus in the Puncture group, which may be caused by the collapse of the intervertebral space and compression of the components within the intervertebral disc, thus making the intervertebral disc denser. After stimulation with bleomycin (Puncture+Bleomycin), faster fibrosis, an increase in the resistance of the disc to deformation, and preservation of the intervertebral space height were simultaneously achieved. Similar results can be seen from statistical analysis which was detailedly shown in FIG. 8.

### Embodiment 6

All animal experiments were approved by the Animal Care and Ethics Committee of the Ninth People's Hospital of Shanghai Jiao Tong University of Medicine, and were conducted according to the principles and procedures of Guide for the Care and Use of Laboratory Animals of the National Institutes of Health (NIH). Six 8-week-old male Sprague-Dawley rats (Shanghai Laboratory, Shanghai Animal Research Center, China) were raised at 26-28° C. and 50-65% humidity for 12 h day and night. Standard rodent foods are fed to rats and the rats have free access to fresh water. Before surgery, the rats were anesthetized by intraperitoneal injection of sodium pentobarbital (5 mg/100 g of body weight). The tails of rats were disinfected with iodinated polyvinyl pyrrolidone, and then a longitudinal skin incision was made to the tail near the ventral part to expose 6-10 parts of the intervertebral disc of the tail bone. The Co6/7 disc was used as a sham control, and Co7/8, Co8/9, Co9/10, and Co10/11 were used as experimental groups. A sterile 20-gauge needle was used to perpendicularly puncture the intervertebral disc through the skin, so as to ensure the needle passed through the annulus fibrosus and stuck into the nucleus pulposus located at the center of the intervertebral disc. The incision was then sutured and the rats were allowed to recover for two weeks. The rats (n=6) were re-subjected to surgery to expose Co8/9, Co9/10, and Co10/11 discs, 10\hat{ }5 L929 cells, 5 μl of 5 ug/ml bleomycin, and a combination of 5 μl of 5 ug/ml bleomycin and 10\hat{ }5 L929 cells were injected into each disc, respectively. The surgical incision was then sutured and the rats were allowed to recover for four weeks. At the end of the experiment, all rats were executed, tails were collected, soft tissues were cleared, and the spines were fixed with 4% PFA. Co6/7 was the control group (sham group), Co7/8 was the Puncture group, Co8/9 was the allogeneic fibroblast treatment group (DFb group), Co9/10 was the bleomycin administration group (Bleomycin group), and Co10/11 was the bleomycin+fibroblast treatment group (Bleomycin+DFb group, Rescure group).

### Embodiment 7

Digital X-ray imaging of the punctured disc samples (Puncture and Rescure groups) prepared in Embodiment 6 was performed in the anterior-posterior axis with a detector that provides up to 5× geometric magnification (Faxitron VersaVision; Faxitron Bioptics LLC, Tucson, AZ. USA). MRI imaging of the same punctured disc samples was performed on a Siemens MagnetomPrisma E11 (Siemens Healthineers, Erlangen, Germany) with the following parameters: TR 3000 ms, TE 80 ms, thickness 1.1 mm, interval 0.22 mm, field of view 160×65 mm, voxel size 0.25×0.1 mm.

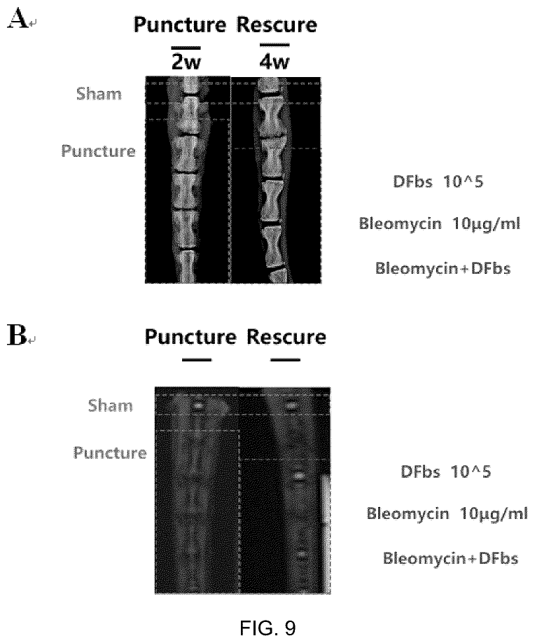

As seen from the digital X-ray imaging results, the intervertebral disc in the Puncture group was severely collapsed with osteophyma formation (shown on the left of FIG. 9A), and the signal of the intervertebral disc in the Puncture group was darkened as seen from the MRI imaging results (shown on the left in FIG. 9B). The digital X-ray and MRI results of the corresponding Rescure group showed that the intervertebral space height could be effectively maintained after bleomycin treatment and fibroblast treatment, and a better therapeutic effect can be realized after bleomycin+fibroblast treatment (shown on the right of FIG. 9A and FIG. 9B).

In summary, the present disclosure effectively overcomes the shortcomings of the prior art and has good transformation prospects and value. The above embodiments are only examples for clearly illustrating the principles and effects of the present disclosure, and are not intended to limit the present disclosure. Any person skilled in the art can modify or change the above embodiments without departing from the spirit and scope of the present disclosure. Therefore, all equivalent modifications or changes made by those who have common knowledge in the technical field without departing from the spirit and technical ideas disclosed by the present disclosure should still be covered by the claims of the present disclosure.

1. Use of an intervertebral disc fibrosis-inducing substance in preparation of medication, wherein the medication is used for the treatment of degenerative spinal diseases.

2. The use according to claim 1, wherein the intervertebral disc fibrosis-inducing substance comprises bleomycin.

3. The use according to claim 2, wherein the intervertebral disc fibrosis-inducing substance comprises a combination of bleomycin and a fibroblast.

4. The use according to claim 3, wherein the fibroblast is a dermal fibroblast; and/or, the fibroblast is an autologous fibroblast.

5. The use according to claim 1, wherein the intervertebral disc fibrosis-inducing substance is capable of inducing the high expression of TGF-β in annulus fibrosus cells.

6. The use according to claim 1, wherein the intervertebral disc fibrosis-inducing substance is capable of upregulating the expression of p-Smad2 and/or p-Samd3 in annulus fibrosus cells.

7. The use according to claim 1, wherein the intervertebral disc fibrosis-inducing substance is capable of upregulating the expression of FSP1 in annulus fibrosus cells;
and/or, the intervertebral disc fibrosis-inducing substance is capable of upregulating the expression of type I collagen in annulus fibrosus cells;
and/or, the intervertebral disc fibrosis-inducing substance is capable of upregulating the expression of type III collagen in annulus fibrosus cells.

8. The use according to claim 1, wherein the degenerative spinal disease is selected from intervertebral disc degeneration.

9. The use according to claim 1, wherein the intervertebral disc fibrosis-inducing substance is the only active ingredient.

10. A composition, comprising an intervertebral disc fibrosis-inducing substance, wherein the intervertebral disc fibrosis-inducing substance comprises a combination of bleomycin and a fibroblast.
